(19) United States
(12) Patent Application Publication
(10) Pub. No.: US 2018/0303995 A1
(43) Pub. Date: Oct. 25, 2018

(54) SYSTEM AND METHOD FOR EXTRACORPOREAL BLOOD TREATMENT

(71) Applicant: Vital Therapies, Inc., San Diego, CA (US)

(72) Inventors: Jan Stange, Rostock (DE); John Brotherton, Cardiff by the Sea, CA (US)

(21) Appl. No.: 15/735,571

(22) PCT Filed: Jun. 14, 2016
(86) PCT No.: PCT/US16/37410

§ 371 Date: Dec. 11, 2017

Related U.S. Application Data
(60) Provisional application No. 62/175,891, filed on Jun. 15, 2015.
(60) Provisional application No. 62/199,821, filed on Jul. 31, 2015.
(60) Provisional application No. 62/199,842, filed on Jul. 31, 2015.

(51) Int. Cl. A61M 1/16; B01D 61/28; B01D 63/02; A61M 1/34; A61M 1/36

(52) U.S. Cl. CPC A61M 1/1698; B01D 61/28; B01D 63/02; A61M 1/3489; B01D 2311/06; A61M 1/3679; A61M 1/3403; A61M 2205/15; A61M 2205/366; A61M 1/3675

(57) ABSTRACT

Provided is an extracorporeal filtration and detoxification system and method generally including separating ultrafiltrate from cellular components of blood, treating the ultrafiltrate independently of the cellular components in a recirculation circuit, recombining treated ultrafiltrate and the cellular components, and returning whole blood to the patient.

### CROSS REFERENCE TO RELATED APPLICATIONS

This application claims the benefit of priority under 35 U.S.C. § 119(e) of U.S. Provisional Patent Application Ser. No. 62/175,891, filed Jun. 15, 2015, U.S. Provisional Patent Application Ser. No. 62/199,821, filed Jul. 31, 2015, and U.S. Provisional Patent Application Ser. No. 62/199,842, filed Jul. 31, 2015, the entire contents of which are incorporated herein by reference in their entireties.

### BACKGROUND OF THE INVENTION

### Field of the Invention

The present invention relates generally to metabolic detoxification, and more particularly to an extracorporeal blood filtration and detoxification system and method employing a recirculation circuit.

### Background Information

The processing of blood has been performed to remove a variety of blood constituents for therapeutic purposes. Examples of blood processing methods include hemodialysis that allows to remove metabolic waste products from the blood of patients suffering from inadequate kidney function. Blood flowing from the patient is filtrated to remove these waste products, and then returned to the patient. The method of plasmapheresis also processes blood using tangential flow membrane separation, to treat a wide variety of disease states. Membrane pore sizes can be selected to remove the unwanted plasma constituents. Blood can be also processed using various devices utilizing biochemical reactions to modify biological constituents that are present in blood. For instance, blood components such as bilirubin or phenols can be gluconized or sulfated by the in vitro circulation of blood plasma across enzymes that are bonded to membrane surfaces.

Various techniques, such as centrifugation, have been available for washing blood cells prior to returning them to the patient. In such techniques a centrifuge is used for separating and washing the red cells in batches. This is a relatively slow process, the apparatus for performing which can be complex and expensive.

Presently used technologies are generally deficient with respect to supporting patients with compromised liver function, for example. Conventional systems and methods suffer from various problems associated with sustaining such patients until a suitable donor organ can be found for transplantation or until the patient's native liver can regenerate to a healthy state.

### SUMMARY OF THE INVENTION

Embodiments of the present invention overcome the foregoing and various other shortcomings of conventional technology, providing an extracorporeal blood filtration and detoxification system and method employing a recirculation circuit.

In accordance with one aspect of the present invention, a system and method provide liver support for multiple therapeutic applications related to acute liver disease, allowing for either the potential regeneration of the impaired or partial liver to a healthy state, or the support of the patient with acute liver failure until all or part of a suitable donor organ can be found for transplant.

Aspects of the present invention provide an extracorporeal blood filtration and detoxification system and method employing an ultrafiltrate generator, a recirculation circuit having an active cartridge including live cells (a bioreactor), and a diffusion component that increases transfer of low molecular weight components from the ultrafiltrate generator into the recirculation circuit for faster clearance and improved treatment.

In one aspect, the invention provides an extracorporeal detoxification system. The system includes: (a) a blood circuit configured to be coupled to a patient and operative to communicate blood from the patient, through an ultrafiltrate generator, and back to the patient; (b) a recirculation circuit coupled to the ultrafiltrate generator and operative to draw ultrafiltrate from the ultrafiltrate generator and to treat ultrafiltrate independently of cellular components of the blood; (c) a conduit junction operative to recombine the ultrafiltrate in the recirculation circuit and the cellular components in the blood circuit prior to reintroduction to the patient; and (d) a diffusion component operative to allow increased transport of low molecular weight substances from the blood into flow of ultrafiltrate within the recirculation circuit. In embodiments, the diffusion component is configured such that flow of ultrafiltrate within the recirculation circuit and blood flow within the ultrafiltrate generator are separated by a semipermeable membrane with the flow of blood and the flow of ultrafiltrate being directed along opposing sides of the semipermeable membrane.

Aspects of the present invention provide extracorporeal blood filtration and detoxification system and method employing an albumin detoxifying component (ADC) and a recirculation circuit having an active cartridge including live cells.

In one aspect, the invention provides an extracorporeal detoxification system. The system includes: (a) a blood circuit configured to be coupled to a patient and operative to communicate blood from the patient, through an ultrafiltrate generator, and back to the patient; (b) a recirculation circuit coupled to the ultrafiltrate generator and operative to draw ultrafiltrate from the ultrafiltrate generator and to treat ultrafiltrate independently of cellular components of the blood; (c) a conduit junction operative to recombine the ultrafiltrate in the recirculation circuit and the cellular components in the blood circuit prior to reintroduction to the patient; and (d) an albumin detoxifying component (ADC) operable to reduce albumin bound toxins and increase albumin binding capacity (ABiC). Aspects of the present invention provide an extracorporeal blood filtration and detoxification system and method employing an ultrafiltrate generator, a recirculation circuit having an active cartridge including live cells (a bioreactor), a citrate infusion port and component to remove citrate from fluid of the system thereby allowing for citrate anticoagulation for improved treatment of patients.

In one aspect, the invention provides an extracorporeal detoxification system. The system includes: (a) a blood circuit configured to be coupled to a patient and operative to communicate blood from the patient, through an ultrafiltrate generator, and back to the patient; (b) a recirculation circuit coupled to the ultrafiltrate generator and operative to draw ultrafiltrate from the ultrafiltrate generator and to treat ultrafiltrate independently of cellular components of the blood; (c) a conduit junction operative to recombine the ultrafiltrate in the recirculation circuit and the cellular components in the blood circuit prior to reintroduction to the patient; (d) a citrate infusion port; and (e) a component operable for removal of citrate. In embodiments, the component is a dialyzer or a device for citrate absorption.

In yet another aspect, the invention provides a method of performing extracorporeal detoxification. The method includes circulating blood of a subject through the device of the present disclosure and returning the blood back to the circulatory system of the subject.

### BRIEF DESCRIPTION OF THE DRAWINGS

FIG. 1 is a simplified block diagram illustrating a prior art extracorporeal filtration and detoxification system.

FIG. 2 is a simplified block diagram illustrating one embodiment of an extracorporeal filtration and detoxification system having an albumin detoxifying component (ADC).

FIG. 3 is a simplified block diagram illustrating one embodiment of an extracorporeal filtration and detoxification system having an albumin detoxifying component (ADC).

FIG. 4 is a simplified block diagram illustrating one embodiment of an extracorporeal filtration and detoxification system having a component operable to remove citrate as disclosed herein.

FIG. 5 is a simplified block diagram illustrating one embodiment of an extracorporeal filtration and detoxification system having a component operable to remove citrate as disclosed herein.

FIG. 6 is a simplified block diagram illustrating one embodiment of an extracorporeal filtration and detoxification system having a diffusion component as disclosed herein.

### DETAILED DESCRIPTION OF THE INVENTION

The present invention is based on innovative concepts for improving performance of human liver cell therapy utilizing a bioartificial liver support system. The present disclosure provides an improved system for filtering and detoxifying blood in providing treatment to a subject requiring extracorporeal blood treatment.

Before the present compositions and methods are further described, it is to be understood that this invention is not limited to the particular systems, methods, and experimental conditions described, as such systems, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The following terminology, definitions and abbreviations apply.

The term “albumin detoxifying component (ADC)” refers to a component that is operable to increase albumin-binding capacity (ABiC). In some embodiments, the ADC includes activated charcoal, such as a filter or column having activated charcoal as described in U.S. Pat. No. 8,236,927, which is incorporated herein by reference in its entirety. The ADC may include dextran or a modified dextran, such as hydroxyalkoxypropyl-dextran optionally substituted with long chain alkyl ethers (e.g., Lipidex-1000™ and Lipophilic Sephadex™ LH-20-100). In some embodiments, the ADC includes a biological component operable to sequester, bind or inactivate albumin bound toxins, such as a bioreactor including cells and/or biological molecules which may be bound to a surface. The ADC may have any suitable configuration, such as a column, canister, filter or the like. An essential advantage of the invention is that the albumin is not substantially changed structurally and retains its native conformation upon flow through the ADC. Thus, following reinfusion into a patient, a considerably higher activity is obtained.

The term “component operable for citrate removal” refers to a device operable to remove citrate from solution, such as a dialyzer or filter including a citrate sequestering agent.

The term “active cartridge” refers to a hollow fiber based cartridge comprising cells (such as, for example, cells of the C3A cell line) having utility in therapeutic applications and detoxification processes.

The term “blood circuit” refers to a circuit of tubing connected to a double lumen catheter and operative to circulate blood from a patient to a blood control unit and back to the patient.

The term “C3A cell line” refers to a sub-clone of the human hepatoblastoma cell line HepG2. In some embodiments, C3A cells may be contained in the extracapillary space of one or more active cartridges. The C3A cell line has been deposited at the American Type Culture Collection under ATCC No. CRL-10741.

The term “detoxification device” refers to a cartridge, canister, or other device that provides a means of removal of specific or non-specific molecules from a fluid stream. Examples would be a dialysis cartridge, an adsorption cartridge, or a filter.

The term “extracapillary space” (ECS) refers to space outside the hollow fibers of active cartridges or an ultrafiltrate generator. The ECS of active cartridges may generally house the C3A cells.

The term “intracapillary space” (ICS) refers to space inside the hollow fibers of active cartridges or an ultrafiltrate generator. The ICS is the flow path for whole blood or the ultrafiltrate fluid.

The term “recirculation circuit” refers to a circuit generally enabling filtration, detoxification, and treatment of ultrafiltrate fluid; in some implementations, a recirculation circuit generally encompasses a reservoir, an oxygenator, and one or more active cartridges.

The term “transmembrane pressure” (TMP)” refers to pressure across the membrane. In particular, within the ultrafiltrate generator or other membranous cartridge, the mean pressure in the ICS minus the mean pressure in the ECS. The amount of ultrafiltration may generally be determined by the TMP across the cartridge membrane; accordingly, TMP and the amount and rate of ultrafiltration may generally be a function of the operational characteristics of an ultrafiltrate pump as well as various physical properties (e.g., pore size and surface area) of the membrane employed in the ultrafiltrate generator.

The term “ultrafiltrate” (UF) refers to plasma fluid and dissolved macromolecules filtered across the semi-permeable membrane of an ultrafiltrate generator.

The term “ultrafiltrate generator” (UFG) refers to a device comprising or embodied as a “blank” active cartridge (i.e., a hollow fiber cartridge which does not contain therapeutically active cells) and operative to separate plasma fluid (ultrafiltrate) from cellular blood components. The hollow fibers may be composed of a semi-permeable membrane which has, for example, a nominal molecular weight cut-off of approximately 100,000 Daltons in some implementations. During use of the UFG, blood may be circulated through the ICS of the hollow fibers; ultrafiltrate, comprising blood plasma and various macromolecules, passes through the membrane fiber walls into the recirculation circuit, where it is circulated through one or more active cartridges.

The term “ultrafiltration” refers generally to a process during which ultrafiltrate is pulled from whole blood across the semi-permeable membrane of the UFG. In some embodiments described below, an ultrafiltrate pump may control the rate of ultrafiltrate production, while the pore size of the hollow fiber membrane of the UFG may control the amount of ultrafiltrate permeating the membrane.

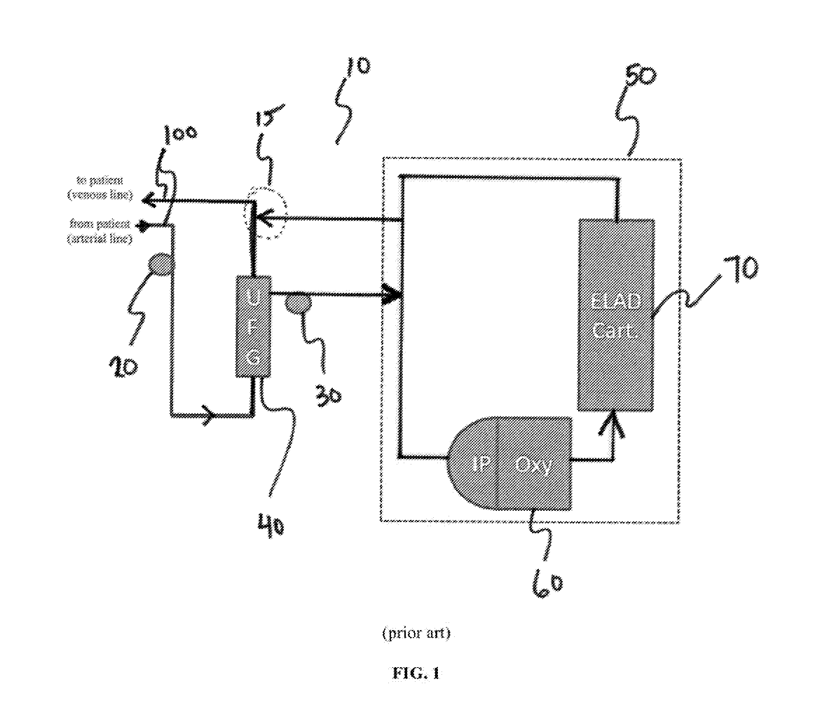

Turning now to the drawings, FIG. 1 is a simplified block diagram illustrating one embodiment of an extracorporeal filtration and detoxification system as described in U.S. Pat. No. 8,105,491, which is incorporated herein by reference in its entirety. As indicated in FIG. 1, system 10 generally includes a blood circuit 100 configured to be coupled to a patient and operative to communicate blood from the patient, through an ultrafiltrate generator (UFG) 40, and back to the patient; a recirculation circuit 50 coupled to the UFG 40 and operative to draw ultrafiltrate from the UFG 40 and to treat ultrafiltrate independently of cellular components of the blood; and a conduit junction 15 operative to recombine the ultrafiltrate in the recirculation circuit 50 and the cellular components in the blood circuit 100 prior to reintroduction to the patient. Also shown in FIG. 1 is an active cartridge 70 and oxygenator 60 arranged within the recirculation circuit 50. The active cartridge 70 is utilized to treat the ultrafiltrate.

The UFG generally includes one or more “blank” hollow fiber cartridges operative to separate UF from cellular components of the whole blood drawn from a patient. Alternative methods can be used for plasma separation, if desired. For example, centrifugation can be used.

The present invention is based in-part on the unexpected finding that reducing the concentration of serum albumin bound molecules in incoming blood plasma from the patient improves the binding capacity of the serum albumin which results in improved performance of human liver cell therapy utilizing a bioartificial liver support system, for instance the system of the invention.

Human serum albumin is found in human blood and is the most abundant protein in human blood plasma constituting about half of serum protein. It is produced in the liver as prepro-albumin and functions to transport various biomolecules such as hormones, fatty acids, and various other compounds including those considered to be toxins. By incorporating an albumin detoxifying component within the blood circuit, albumin-binding capacity (ABiC) may be increased resulting in reduced toxicity in blood returned to the patient. Additionally, detoxifying serum albumin before ultrafiltrate enters the active cartridge results in increased cell count, growth and viability of cells residing in active cartridge 70 which also results in increased production of therapeutic factors, e.g., secretory proteins, by such cells.

ABiC is a measure for characterizing the site-specific binding functions of the albumin molecule. As discussed herein, it was determined that reduced ABiC in liver failure is linked to an increase in albumin-bound toxins. As discussed further in Example 1, experiments using primary hepatocytes showed that a reduction of albumin bound toxins and improvement of ABiC resulted in reduced cell death and corresponding increase in cell viability.

Accordingly, in one aspect, the system includes: (a) a blood circuit configured to be coupled to a patient and operative to communicate blood from the patient, through an ultrafiltrate generator, and back to the patient; (b) a recirculation circuit coupled to the ultrafiltrate generator and operative to draw ultrafiltrate from the ultrafiltrate generator and to treat ultrafiltrate independently of cellular components of the blood; (c) a conduit junction operative to recombine the ultrafiltrate in the recirculation circuit and the cellular components in the blood circuit prior to reintroduction to the patient; and (d) an ADC operable to reduce albumin bound toxins and increase ABiC.

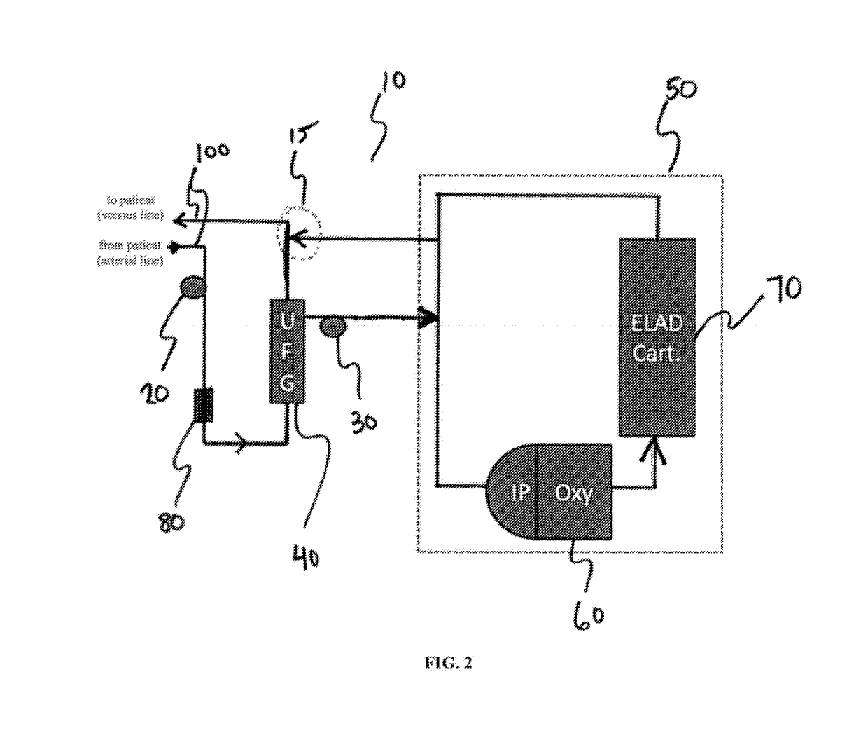

FIG. 2 is a simplified block diagram illustrating one embodiment of system 10 which includes an ADC 80 disposed upstream of active cartridge 70 and downstream of UFG 40.

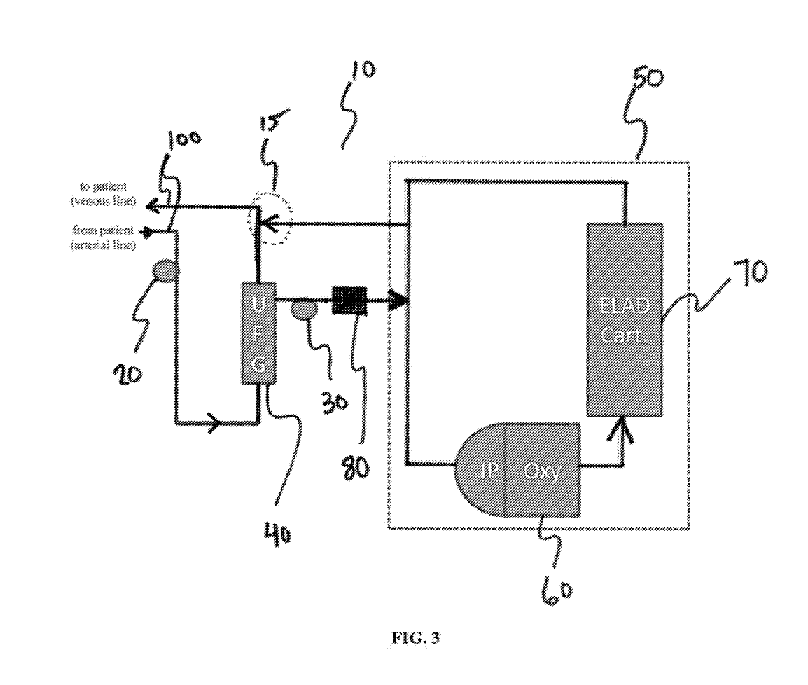

FIG. 3 is a simplified block diagram illustrating one embodiment of system 10 which includes an ADC 80 disposed downstream of UFG 40 and recirculation circuit 50.

While the embodiments of FIGS. 2 and 3 include ADC 80 downstream of active cartridge 70, it is envisioned that ADC 80 may be disposed at any point along the blood circuit, for example, downstream of active cartridge 70 in recirculation circuit 50 or downstream of recirculation circuit 50. In one embodiment, ADC 80 is disposed in recirculation circuit 50 downstream of oxygenator 60 and upstream of active cartridge 70, or upstream of both oxygenator 60 and active cartridge 70.

In embodiments in which the system includes an ADC, the ADC is operable to increase ABiC by at least about 5, 10, 15, 20, 25, 30, 35, 40, 45, 50, 55, 60, 65, 70, 75, 80, 85, 90, 95, 100, 150, 200, 300, 400, 500, 1,000% or greater as compared to ABiC before flow through the ADC. In some embodiments, ABiC is increase by a factor of at least about 1.5, 2.0, 3.0, 4.0, 5.0, 10, 20, 30, 40, 50, 75, 100, 250, 500, 1000, 5000, 10,000 or greater as compared to ABiC before flow through the ADC. Additionally, the ADC is operable to reduce the total concentration of bile acids to less than about 100, 90, 80, 70, 60, 50, 40, 30, 20, 15, 10, 5 or 1 μmol/l or less.

ABiC may be measured using any method known in the art. In one embodiment, the determining method is based on the estimation of the unbound fraction of a specific albumin-bound marker in a plasma sample. By comparing it with the fraction of unbound marker in a reference albumin solution, the site-specific binding capacity of the sample can be expressed semi-quantitatively. The ABiC in the context of this invention is determined as described in Klammt et al. (Z Gastroenterol 39:24-27 (2001)). Firstly, the albumin concentration in an albumin solution is determined by scattering measurements (nephelometry) and the solution is then adjusted to an albumin concentration of 150 μmol/l or 300 μmol/l by dilution. Next, one volume of the albumin solution with a predetermined concentration of a fluorescence marker (dansylsarcosin, Sigma Chemical) which is specific for binding site II (diazepam binding site) of the albumin is added in an equilmolar ratio and incubated for 20 min at 25° C. After incubation, unbound fluorescence marker is separated out by ultrafiltration (Centrisart I, Sartorius Gottingen; exclusion size: 20000 dalton) and the amount of unbound fluorescence marker in the separated solution is determined by fluorescence spectrometry (Fluoroscan, Labsystems, Finland; excitation: 355 nm; emission: 460 nm). To reinforce the fluorescence, the solution of unbound fluorescence marker is supplemented with ligand-free albumin (fatty acid free; from Sigma Aldrich in powder form) in a concentration of 150 μmol/l or 300 μmol/l. Alongside the sample amino acid solution, the same measurement is carried out on a corresponding solution of a reference albumin. The reference is purified and deligandised human serum albumin (BiSeKo, Biotest Pharma GmbH, Dreieich, Germany). Alternatively, the albumin can also be removed from a serum pool of more than 50 healthy blood donors (using Deutsches Rotes Kreuz [German red Cross] criteria). The ABiC is calculated using the following formula:

$$ABiC ( \% ) = \frac{fluorescence in the filtrate of the reference}{fluorescence in the filtrate of the sample} * 100. \qquad ( Equation 1 )$$

The ABiC measured in accordance with Klammt et al. and using the above formula does not give the absolute binding capacity of albumin for all of its binding sites, but the relative binding capacity, compared with the reference albumin, for ligands which bind to Sudlow II binding sites (diazepam binding sites). It can thus have a value of more than 100%. The special measurement method is, however, particularly suitable for measuring even the smallest changes in the ABiC as the marker is particularly easily expelled from the bond.

An essential advantage of the method of the invention is that the albumin is not substantially changed structurally under extreme conditions such as severe acidification or the use of denaturing means, but essentially retains its native conformation. Thus, following infusion into a patient, and due to the improved binding capacity, a considerably higher activity is obtained.

In one embodiment of the invention, the ABiC of the blood fraction produced by the ADC, measured in accordance with Klammt et al., is at least 60%, preferably at least 70%, particularly preferably at least 80% and more particularly preferably at least 90%.

In a particularly preferred implementation of the invention, the ADC includes activated charcoal. The activated charcoal is advantageously used as a material which can form a suspension or as a powder, for example packed in a column or as a bed of adsorption material. It is important that the activated charcoal particles in the powder can form channels between the particles which on the one hand are sufficiently large to allow the albumin solution to flow through the adsorption material with a sufficient flow rate, and on the other hand are sufficiently narrow that the albumin molecules in the albumin solution can come into direct surface contact with the activated charcoal particles at a high frequency during flow through.

Alternatively, the activated charcoal can also be embedded as the adsorption material in a solid porous matrix, for example a polymer matrix formed from cellulose, resin or other polymer fibers or open-pored foams. When embedding the activated charcoal in a matrix, care should be taken that the matrix allows the albumin solution to flow in and that the matrix carries the activated charcoal particles in such a manner that they can come into contact with the albumin solution. Further, the porosity of the matrix material should be such that the pores can form channels to allow the albumin solution to flow through.

In one embodiment, a support matrix with hydrophilic properties is used, which allows the adsorption material to be wetted. Such a support matrix can, for example, include cellulose or other natural or synthetically produced hydrophilic polymers.

Activated charcoal itself is a porous material which within its particle has macropores (>25 nm), mesopores (1-25 nm) and micropores (<1 nm), so that the activated charcoal has a very large internal surface area. The size of these pores is normally given for activated charcoal by the molasses number (macropores), the methylene blue adsorption (mesopores) and the iodine number (micropores). The internal surface area is determined using BET and given in m2/g activated charcoal. Activated charcoal is generally known as an adsorption medium which takes molecules into its pores and retains them therein or immobilizes substances by surface bonds. Because of the high porosity and internal surface area, activated charcoal has a very high adsorption capacity compared with its weight or external volume. This is dependent on the molecules being able to diffuse into these pores.

In embodiments, the activated charcoal is selected so that it has a molasses number (IUPAC) of 100 to 400, preferably 200 to 300. It may also have a methylene blue adsorption (IUPAC) of 1 to 100 g/100 g of activated charcoal, preferably 10 to 30 g/100 g of activated charcoal, an iodine number (IUPAC) of 500 to 3000, preferably 800 to 1500, and/or a total internal surface area (BET) (IUPAC) of 100 to 5000 m2/g of activated charcoal, preferably 800 to 1400 m2/g activated charcoal.

As discussed herein, active cartridge 70 includes live cells which continuously secrete therapeutic factors in the UF passing through the cartridge. Cells of the active cartridge 70 depend on sufficient ionized calcium for functionality. Citrate functions as an anti-coagulant in the bloodstream by binding ionized calcium (e.g., calcium cation). As such, conventional systems are limited to systemic anticoagulation (e.g., systemically within the patient) versus regional anticoagulation within the system. Systematic anticoagulants bare the risk of bleeding or insufficient anticoagulation and therefore premature clotting, which affects the risk benefit ratio of such therapies negatively.

With reference to FIG. 1, the current modus of anticoagulation is based on systemic anticoagulants given to the patient, either systemically or in the incoming arterial line from the patient. In the case of heparin, between 100 and 2000 Units/hour are infused which leads to a systemic prolongation of the activated partial thromboplastin time (aPtt), increasing the bleeding risk to the patient. As such, the problem is that UF used to form blood flowing back in to the patient is incapable of coagulating.

The present invention addresses this problem and provides a system in which regional anticoagulation within the system is achieved using citrate. For example, citrate is applied into the blood circuit 100 of system 10 and subsequently removed from UF before the UF is reintroduced to the patient. Ionized calcium is also introduced in the UF upon removal of the citrate and reintroduction of the UF into the patient.

Accordingly, in one aspect, the system includes: (a) a blood circuit configured to be coupled to a patient and operative to communicate blood from the patient, through an ultrafiltrate generator, and back to the patient; (b) a recirculation circuit coupled to the ultrafiltrate generator and operative to draw ultrafiltrate from the ultrafiltrate generator and to treat ultrafiltrate independently of cellular components of the blood; (c) a conduit junction operative to recombine the ultrafiltrate in the recirculation circuit and the cellular components in the blood circuit prior to reintroduction to the patient; (d) a citrate infusion port; and (e) a component operable for removal of citrate. In embodiments, the component is a dialyzer or a device for citrate absorption.

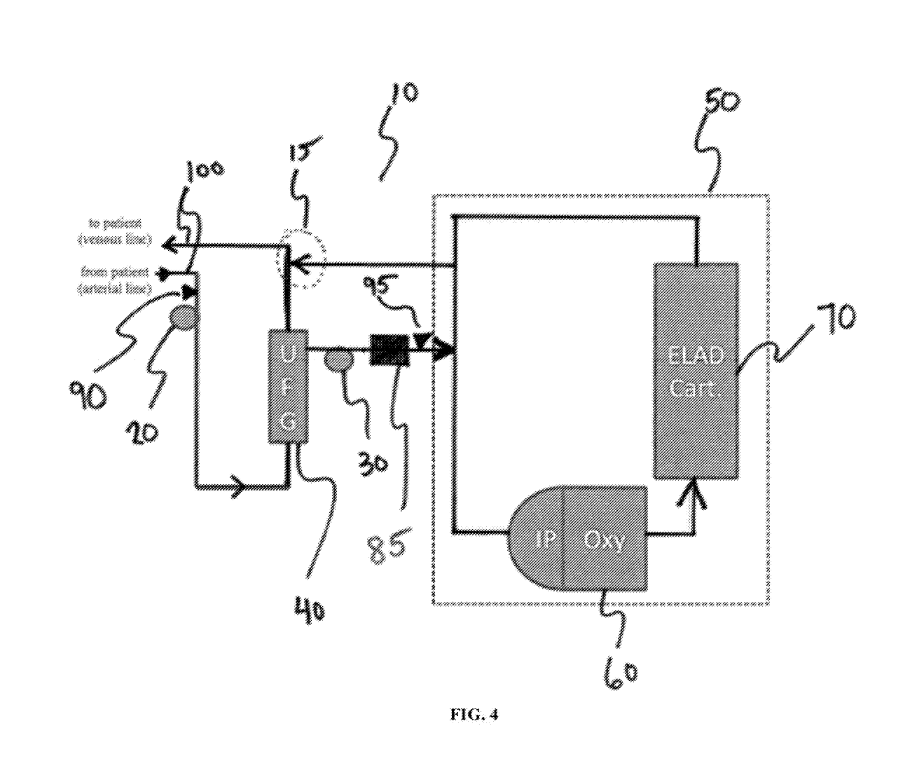

FIG. 4 is a simplified block diagram illustrating one embodiment system 10 which includes a citrate infusion port 90 and a component operable for citrate removal 85 (shown as a dialyzer in this embodiment). The system of FIG. 2 also includes an ionized calcium infusion port 95 downstream of component 85 and upstream of active cartridge 70 so that calcium may be reintroduced to UF before UF is passed through the active cartridge.

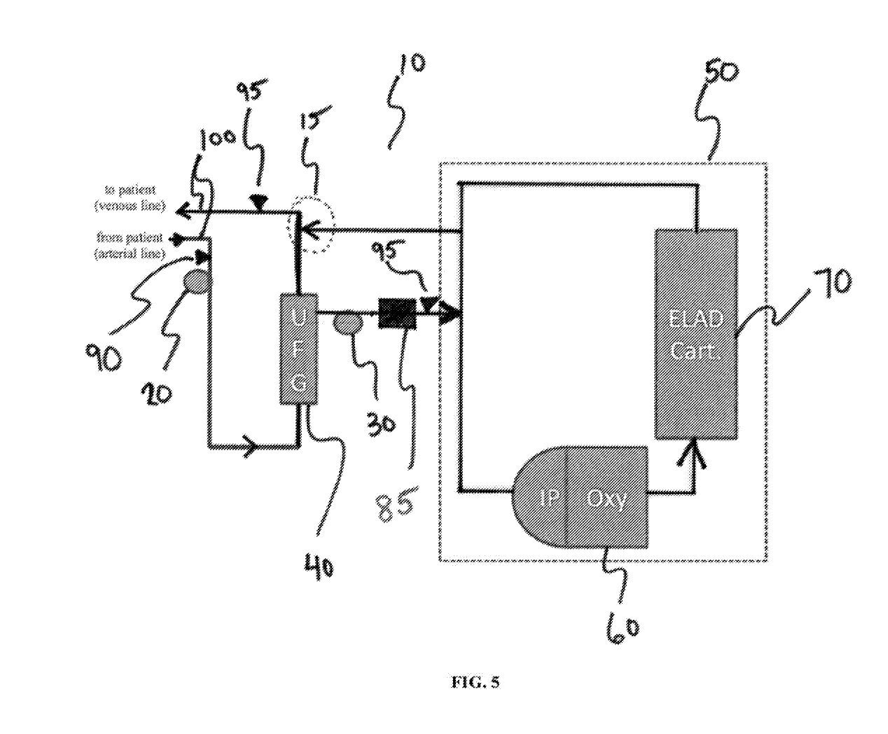

FIG. 5 is a simplified block diagram illustrating one embodiment system 10 which includes a citrate infusion port 90 and a component operable for citrate removal 85 (shown as a dialyzer in this embodiment). The system of FIG. 5 also includes a first ionized calcium infusion port 95 downstream of component 85 and upstream of active cartridge 70 and a second ionized calcium infusion port 95 downstream of active cartridge 70 to replenish ionized calcium which was depleted by cells of the active cartridge.

In various embodiments, system 10 may include one or more infusion ports for infusion of cations other than calcium, for example, magnesium, which may also complex with citrate anion and which must be replenished in UF after removal of citrate by component 85.

In various embodiments, system 10 may include one or more calcium and/or citrate sensors disposed along blood circuit 100 to monitor ionized calcium and/or citrate concentrations. The sensors may be configured to detect levels of ionized calcium, citrate, and/or calcium citrate. In one embodiment, system 10 includes a sensor adjacent each citrate or calcium infusion port along blood circuit 100. For example, a calcium sensor may be located downstream of calcium infusion port 95 and upstream of active cartridge 70. Additionally, a calcium sensor may be located downstream of active cartridge 70 and downstream of junction 15.

As used herein, “citrate” refers to a citrate anion, in any form, including citric acid (citrate anion complexed with three protons), salts containing citrate anion, and partial casters of citrate anion. Citrate anion is an organic tricarboxylate. Citric acid, which has been assigned Chemical Abstracts Registry No. 77-92-2, has the molecular formula HOC(CO₂H)(CH₂CO₂H)₂ and a formula weight of 192.12 g/mol. A citrate salt (i.e., a salt containing citrate anion) is composed of one or more citrate anions in association with one or more physiologically-acceptable cations. Exemplary physiologically-acceptable cations include, but are not limited to, protons, ammonium cations and metal cations. Suitable metal cations include, but are not limited to, sodium, potassium, calcium, and magnesium, where sodium and potassium are preferred, and sodium is more preferred. A composition containing citrate anion may contain a mixture of physiologically-acceptable cations.

Citrate is typically in association with protons and/or metal cations, e.g., calcium or magnesium, upon removal from UF. Exemplary of such citrate compounds are, without limitation, citric acid, sodium dihydrogen citrate, disodium hydrogen citrate, trisodium citrate, trisodium citrate dihydrate, potassium dihydrogen citrate, dipotassium hydrogen citrate, calcium citrate, and magnesium citrate.

In one embodiment sodium citrate provides the source for the citrate anions infused into UF. Sodium citrate may be in the form of a dry chemical powder, crystal, pellet or tablet. Any physiologically tolerable form of citric acid or sodium citrate may be used to introduce citrate anions into UF. For instance, the citric acid or sodium citrate may be in the form of a hydrate, including a monohydrate.

In various embodiments, system 10 is configured to maintain an ionized calcium level in the UF entering the active cartridge at greater than about 0.4, 0.5, 0.6, 0.7, 0.8, 0.9, 1.0, 1.1, 1.2 mmol/l or higher. In one embodiment, system 10 is configured to maintain an ionized calcium level in the UF entering the active cartridge at greater than about 0.8 mmol/l. In various embodiments, system 10 is configured to maintain an ionized calcium level in fluid downstream of junction 15 at less than about 0.9, 0.8, 0.7, 0.6, 0.5, 0.4, 0.3, 0.2 mmol/l or less. In various embodiments, system 10 is configured to maintain an ionized calcium level in fluid downstream of junction 15 at less than about 0.5 mmol/l or less.

In various embodiments, citrate is infused into UF before UFG 40 in an amount of up to about 0.5, 0.6, 0.7, 0.8, 0.9, 1.0, 1.1, 1.2, 1.3, 1.4, 1.5, 1.6, 1.7, 1.8, 1.9 or 2.0 moles/L. Calcium and magnesium salts are infused into UF post citrate removal component 85 to keep ionized calcium and magnesium balance.

Current detoxification systems generate UF coming from approximately 100 kD filters. As such, metabolization by cells of an active cartridge of low molecular weight substances present in a patient's blood, such as toxins, is limited to the ultrafiltration rate, for example, the rate at which ultrafiltrate is generated from incoming blood from a patient. In practice utilizing systems such as that disclosed in U.S. Pat. No. 8,105,491 and shown in FIG. 1, the ultrafiltration rate is between about 10 to 60 ml/min.

In systems such as the one represented in FIG. 1, the plasma fraction filtered through UFG 40 enters recirculation circuit 50 which is essentially a closed loop bioreactor, wherein the filtrate flows through the active cartridge 70 with the same flow rate as the flow into and out of recirculation circuit 50. As such, the maximum clearance for toxins in the UF, even if metabolized at 100% in active cartridge 70, is 10 to 60 ml/min. For some toxins, such as ammonia or lactate, those clearances are insufficient to support a liver for detoxification.

The system of the present invention addresses this problem and is based on the unexpected finding that the active cartridge 70 is very effective in removing small molecules of low molecular weight, such as toxins, and that the liver clearance for toxins can be higher than 60 ml/min as a result.

Accordingly, in one aspect, the system includes: (a) a blood circuit configured to be coupled to a patient and operative to communicate blood from the patient, through an ultrafiltrate generator, and back to the patient; (b) a recirculation circuit coupled to the ultrafiltrate generator and operative to draw ultrafiltrate from the ultrafiltrate generator and to treat ultrafiltrate independently of cellular components of the blood; (c) a conduit junction operative to recombine the ultrafiltrate in the recirculation circuit and the cellular components in the blood circuit prior to reintroduction to the patient; and (d) a diffusion component operative to allow increased transport of low molecular weight substances from the blood into flow of ultrafiltrate within the recirculation circuit.

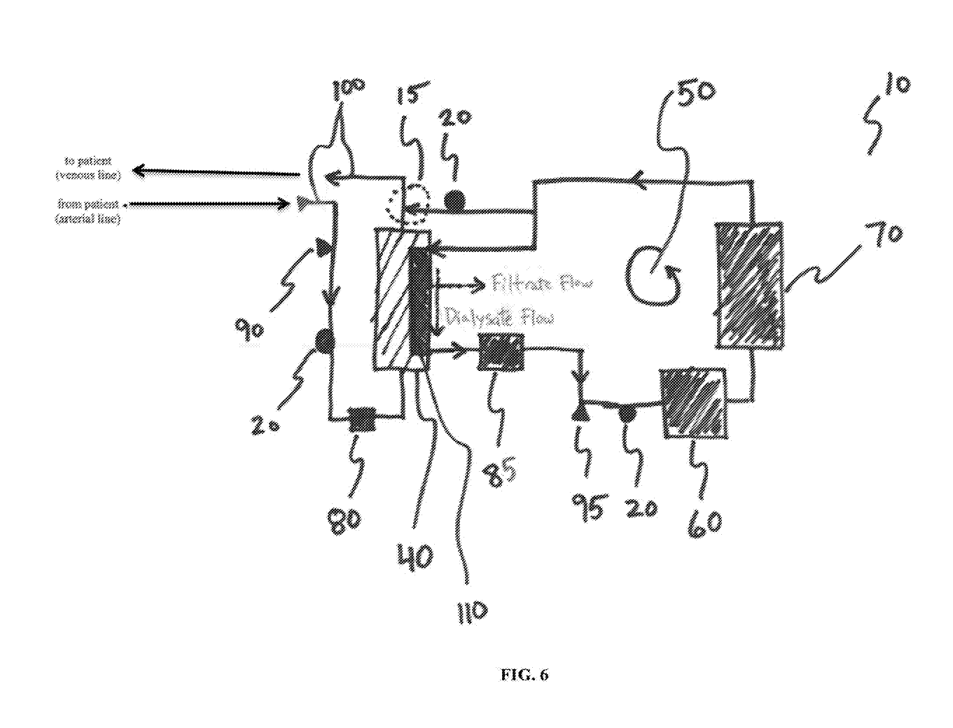

In the present system 10, the limit for mass transfer for low molecular weight substances is increased by including a diffusion component 110 within UFG 40. FIG. 6 is a simplified block diagram illustrating one embodiment of system 10 of the present invention which also includes diffusion component 110 which is operative to allow increased transport of low molecular weight substances from the blood flowing through UFG 40 into the flow of UF passing through recirculation circuit 50 which passes through active cartridge 70.

In embodiments, UFG 40 and diffusion component 110 are configured such that flow of UF within the recirculation circuit 50 and blood flow within UFG 40 are separated by a semipermeable membrane with the flow of blood and the flow of UF being directed along opposing sides of the semipermeable membrane. The flow of UF in recirculation circuit 50 through diffusion component 110 along the membrane may be parallel or counter to the flow of the blood through UFG 40 as shown in FIG. 6. In embodiments, the flow of UF along the membrane (dialysate flow) through diffusion component 110 is greater than the flow of UF being generated across the membrane (filtrate flow) entering recirculation circuit 50. For example, the flow of UF along the membrane in the recirculation circuit 50 may be at least 2, 3, 4, 5, 6, 7, 8, 9, 10, 20, 30, 40, 50, 60, 70, 80, 90, 100 times or greater than that of flow of UF being generated across the membrane.

In this configuration, the maximum clearance for substances out of blood is not limited by the rate of UF via UFG 40. For low molecular weight substances with 100% permeability through the diffusion component membrane (sieving 100%) and which may be 100% removed by cells in active cartridge 70, the clearance in this disclosed embodiment is only limited by blood flow, which can be greater than 60 ml/min, for example, about 70, 80, 90, 100, 110, 120, 130, 140, 150, 160, 170, 180, 190, 200, 210, 220, 230, 240, 250, 500, 100, 1500, 2000, 2500, 3000 ml/min or higher, depending on blood flow.

In embodiments, system 10 is configured such that flow into UFG 40 is about 150-250 ml/min, flow through recirculation circuit 50 is about 1500 to 2500 ml/min and flow downstream of junction 15 is about 10 to 60 ml/min. This is achieved via blood pumps 20 as shown in FIG. 6.

In embodiments, the diffusion component 110 includes a hollow fiber filter having a semi-permeable membrane with a predetermined molecular weight cut-off. In some embodiments, the semi-permeable membrane has a predetermined molecular weight cut-off of less than about 10,000 Daltons, such as 9,000, 8,000, 7,000, 6,000, 5,000, 4,000, 3,000, 2,000, 1,000, 500 or 100 Daltons. In some embodiments, the semi-permeable membrane has a pore size of less than about 0.1, 0.05, 0.01, 0.005, 0.001, 0.0005 or 0.0001 μm.

As used herein, a low molecular weight substance is a substance of less than about 10,000 Daltons, such as 9,000, 8,000, 7,000, 6,000, 5,000, 4,000, 3,000, 2,000, 1,000, 500 or 100 Daltons.

In one aspect, the system includes: (a) a blood circuit configured to be coupled to a patient and operative to communicate blood from the patient, through an ultrafiltrate generator, and back to the patient; (b) a recirculation circuit coupled to the ultrafiltrate generator and operative to draw ultrafiltrate from the ultrafiltrate generator and to treat ultrafiltrate independently of cellular components of the blood; (c) a conduit junction operative to recombine the ultrafiltrate in the recirculation circuit and the cellular components in the blood circuit prior to reintroduction to the patient; and one more of: a diffusion component operative to allow increased transport of low molecular weight substances from the blood into flow of ultrafiltrate within the recirculation circuit, an albumin detoxifying component (ADC) operable to reduce albumin bound toxins and increase ABiC, and a citrate infusion port and component operable for removal of citrate.

In one aspect, the system includes: (a) a blood circuit configured to be coupled to a patient and operative to communicate blood from the patient, through an ultrafiltrate generator, and back to the patient; (b) a recirculation circuit coupled to the ultrafiltrate generator and operative to draw ultrafiltrate from the ultrafiltrate generator and to treat ultrafiltrate independently of cellular components of the blood; (c) a conduit junction operative to recombine the ultrafiltrate in the recirculation circuit and the cellular components in the blood circuit prior to reintroduction to the patient; (d) a diffusion component operative to allow increased transport of low molecular weight substances from the blood into flow of ultrafiltrate within the recirculation circuit; and (e) an albumin detoxifying component (ADC) operable to reduce albumin bound toxins and increase ABiC.

In one aspect, the system includes: (a) a blood circuit configured to be coupled to a patient and operative to communicate blood from the patient, through an ultrafiltrate generator, and back to the patient; (b) a recirculation circuit coupled to the ultrafiltrate generator and operative to draw ultrafiltrate from the ultrafiltrate generator and to treat ultrafiltrate independently of cellular components of the blood; (c) a conduit junction operative to recombine the ultrafiltrate in the recirculation circuit and the cellular components in the blood circuit prior to reintroduction to the patient; (d) a diffusion component operative to allow increased transport of low molecular weight substances from the blood into flow of ultrafiltrate within the recirculation circuit; (e) an albumin detoxifying component (ADC) operable to reduce albumin bound toxins and increase ABiC; (f) and a citrate infusion port; and (g) component operable for removal of citrate.

In one aspect, the system includes: (a) a blood circuit configured to be coupled to a patient and operative to communicate blood from the patient, through an ultrafiltrate generator, and back to the patient; (b) a recirculation circuit coupled to the ultrafiltrate generator and operative to draw ultrafiltrate from the ultrafiltrate generator and to treat ultrafiltrate independently of cellular components of the blood; (c) a conduit junction operative to recombine the ultrafiltrate in the recirculation circuit and the cellular components in the blood circuit prior to reintroduction to the patient; (d) a diffusion component operative to allow increased transport of low molecular weight substances from the blood into flow of ultrafiltrate within the recirculation circuit; (e) and a citrate infusion port; and (f) component operable for removal of citrate.

In one aspect, the system includes: (a) a blood circuit configured to be coupled to a patient and operative to communicate blood from the patient, through an ultrafiltrate generator, and back to the patient; (b) a recirculation circuit coupled to the ultrafiltrate generator and operative to draw ultrafiltrate from the ultrafiltrate generator and to treat ultrafiltrate independently of cellular components of the blood; (c) a conduit junction operative to recombine the ultrafiltrate in the recirculation circuit and the cellular components in the blood circuit prior to reintroduction to the patient; (d) an albumin detoxifying component (ADC) operable to reduce albumin bound toxins and increase ABiC; (e) and a citrate infusion port; and (f) component operable for removal of citrate.

The system 10 of the invention may also include a heparin infusion pump which may be used to introduce heparin into the whole blood upstream of the UFG. Those of skill in the art will appreciate that heparin, an acidic mucopolysaccharide, or various derivatives thereof may provide anticoagulant effects; other anticoagulant agents may be appropriate depending upon, among other things, the nature of the detoxification treatment and various other system parameters.

As set forth above, heparin infusion pump may provide heparin or a similar anticoagulation agent to the blood circuit upstream of the UFG; similarly, a glucose infusion pump may provide a supply of glucose to the UF upstream of recirculation circuit to nourish the C3A or other active cells. In some embodiments, the pumps may include suitable sensors or sensor inputs, actuators, and control electronics operative in accordance with sensor output and control signals to adjust flow rates dynamically as a function of overall flow rate through blood circuit and recirculation circuit, respectively. Indications of overall flow rate may be obtained, for example, from output provided by flow rate or pressure sensors distributed at various locations in the circuits.

In embodiments, air detectors may be implemented to detect air bubbles or other gaseous contaminants within the circulating fluids. In some embodiments, for example, one or more air detectors may be incorporated into the blood circuit, and one or more additional air detectors may also be incorporated at selected locations in the recirculation circuit. As is generally known and practiced in the art, numerous suitable mechanical filtration systems may be employed to remove unwanted gaseous contamination. In some embodiments, one or more of such filtration systems may be selectively operative responsive to output from one or more air detectors. Accordingly, while representation of some of the hardware has been omitted for clarity, it will be appreciated that the present disclosure contemplates detection and removal of air and other gaseous bubbles from the fluidic system, particularly at or near venous access to the patient.

During clinical or therapeutic treatment, UF may be pumped through the lumen (ICS) of the hollow fiber cartridge, allowing toxins, nutrients, glucose, and dissolved oxygen from the UF to diffuse across the membrane into the ECS, where the active cells may metabolize them. Metabolites, along with albumin and other proteins produced by the cells, may diffuse back across the membrane into the UF for return to the patient.

As set forth above and contemplated herein, the C3A cell line may be a subclone of the human hepatoblastoma cell line HepG2. Some subclones of this parent cell line, such as C3A, for example, exhibit liver-specific functional capabilities such as high albumin production and α-fetoprotein (AFP) production. The C3A cell line has demonstrated such liver-specific functionality, and has been described herein by way of example only, and not by way of limitation. In that regard, it is noted that the utility of the system of the present invention, and the respective components thereof is described herein only by way of example; those of skill in the art will recognize that the disclosed system and method may facilitate detoxification and therapeutic treatment in contexts other than liver therapies. The present disclosure is not intended to be limited to any specific application implementing any particular cell line.

In some embodiments, hollow fibers of the active cartridge may have a nominal molecular weight cut-off of greater than 70,000 Daltons, for example, allowing middle molecular weight molecules such as albumin to cross the membrane. Macromolecules produced by the C3A or other active cells may be able to diffuse into the UF circulating through the ICS; similarly, albumin-carrying toxins are able to diffuse from the ICS to the active cells occupying the ECS.

As set forth above, a heparin infusion pump may provide heparin or a similar anticoagulation agent to the blood circuit upstream of UFG 40; similarly, a glucose infusion pump may provide a supply of glucose to the UF upstream of recirculation circuit 50 to nourish the C3A or other active cells.

In embodiments, blood pumps 20 may comprise suitable sensors or sensor inputs, actuators, and control electronics operative in accordance with sensor output and control signals to adjust flow rates dynamically as a function of overall flow rate through blood circuit 100 and recirculation circuit 50. Indications of overall flow rate may be obtained, for example, from output provided by flow rate or pressure sensors distributed at various locations in the circuits substantially as set forth below.

Blood withdrawal pressure may be measured in blood circuit 100. The blood withdrawal monitors fluid pressure and any pressure fluctuations of the outflow of blood from a patient to blood pump 20.

The recirculation circuit 50 generally includes a blood pump 20, an oxygenator 60 and one or more active cartridges 70. The recirculation circuit may optionally contain one or more additional detoxification devices. If desired, the locations of oxygenator 60 and active cartridge 70 can be optionally switched.

Oxygenator 60 may comprise, or be embodied in, any of various membrane oxygenators generally known in the art or other types of oxygenators developed and operative in accordance with known principles. In operation, oxygenator 60 may provide oxygen for utilization in the detoxification or therapeutic process executing in active cartridge 70.

As set forth above, recirculation circuit 50 may incorporate one or more active cartridges 70, each of which may be embodied as or comprise a hollow fiber filter. Accordingly, each active cartridge 70 may comprise a bundle of hollow fibers employing a semi-permeable membrane. Surrounding these fibers, one or more types of active cells may be utilized to treat the UF in a selected manner as the UF circulates through the cartridge. The character, quantity, density, and genetic composition of active cells facilitating treatment in active cartridges 70 may be selected as a function of the overall functionality of system 10 in which recirculation circuit 50 is employed. As set forth herein, an exemplary embodiment of system 10 and recirculation circuit 50 incorporates C3A cells, though other alternatives exist, depending upon, inter alia, the desired utility of system 10 and the nature of the contaminant sought to be removed or treated.

During operation of system 10, UF from circuit 50 may pass through one or more additional filters or filter series prior to reintroduction to the blood circuit 100.

Recirculation circuit 50 may further comprise various other components, such as valve assemblies, for example, to prevent back-flow and provide regulated flow rates on the suction side and the pressure side, respectively, of a recirculation pump. Some embodiments may employ dynamically activated valve assemblies, which may be selectively adjusted to control flow rates precisely; appropriate sensors, such as temperature, pressure, or flow meters and associated electronics and control hardware are not shown in the embodiments of the Figures for clarity. Those of skill in the art will appreciate that various techniques and flow control apparatus are generally known and encompassed herein.

In operation, oxygenator 60 may be positioned within the recirculation circuit at a point upstream of active cartridge 70 to assure that sufficient oxygen is provided to the active cells during therapy. It will be appreciated that an gas flow meters (not shown) may be coupled between the gas supplies and oxygenator 60; as is generally known in the art, such gas flow meters may facilitate regulation of the amount of oxygen delivered to oxygenator 60, ensuring sufficient oxygenation to sustain the therapeutically active cells maintained in active cartridge 70.

System 10 may be designed to provide continuous treatment; accordingly, one or more auxiliary batteries or other uninterruptible power supplies may be provided at various locations in system 10.

Returning whole blood to the patient may involve utilizing valve assemblies or otherwise regulating the flow rate in accordance with the patient's physical condition and internal blood pressure requirements. The system 10 may employ or comprise some or all of the following features or hardware downstream of junction 15: dynamically adjustable valve assemblies enabling precise pressure control or flow regulation; safety valves or back flow restrictors preventing upstream pressure variations from reversing the direction of blood flow; and gas bubble detection and removal apparatus or devices.

With respect to data acquisition and analysis, one or more in-line blood gas analyzers may be implemented upstream or downstream (or both) of active cartridge 70. Where two gas analyzers are arranged upstream and downstream, oxygen and pH differentials from both the upstream and downstream sides of active cartridge 70 may provide important measurements of therapeutic cell function over time during therapy. Such measurements may be made in real-time using multiple in-line analyzers.

The following examples are provided to further illustrate the embodiments of the present invention, but are not intended to limit the scope of the invention. While they are typical of those that might be used, other procedures, methodologies, or techniques known to those skilled in the art may alternatively be used.

### Example 1

### Increase of Albumin Binding Capacity

Experiment: Primary hepatocytes were incubated with heparinized plasma of patients with liver failure before and after treatment with charcoal filters in order to reduce albumin bound toxins and in order to increase ABiC.

Reduction of albumin bound toxins and improvement of ABiC resulted in reduced formation of lipid droplets (as an indicator of mitochondrial dysfunction), reduced “blebbing” as an indicator of apoptosis due to elevated intracellular calcium and improved viability as shown by a “Live Dead” test.

Primary hepatocytes were incubated with heparinized plasma of patients with liver failure before and after treatment with charcoal filters in order to reduce albumin bound toxins and in order to increase ABiC. “Foamy” cells were observed before treatment indicating “blebbing”, a light microscopy symptom of apoptosis and multiple lipid droplets within the hepatocytes. Reducing albumin bound toxins and thereby improving ABiC in the plasma was observed to prevent those symptoms (data not shown).

The present invention has been illustrated and described in detail with reference to particular embodiments by way of example only, and not by way of limitation. Those of skill in the art will appreciate that various modifications to the described exemplary embodiments are within the scope and contemplation of the present disclosure. Therefore, it is intended that the invention be considered as limited only by the scope of the appended claims.

1. An extracorporeal detoxification system, comprising:
(a) a blood circuit configured to be coupled to a patient and operative to communicate blood from the patient, through an ultrafiltrate generator, and back to the patient;
(b) a recirculation circuit coupled to the ultrafiltrate generator and operative to draw ultrafiltrate from the ultrafiltrate generator and to treat ultrafiltrate independently of cellular components of the blood;
(c) a conduit junction operative to recombine the ultrafiltrate in the recirculation circuit and the cellular components in the blood circuit prior to reintroduction to the patient; and
(d) a diffusion component operative to allow increased transport of low molecular weight substances from the blood into flow of ultrafiltrate within the recirculation circuit.

2. The system of claim 1, wherein the diffusion component is configured such that flow of ultrafiltrate within the recirculation circuit and blood flow within the ultrafiltrate generator are separated by a semipermeable membrane with the flow of blood and the flow of ultrafiltrate being directed along opposing sides of the semipermeable membrane.

3. The system of claim 2, wherein the flow of ultrafiltrate along the membrane is parallel or counter to the flow of the blood.

4. The system of claim 2, wherein the flow of ultrafiltrate along the membrane is greater than the flow of ultrafiltrate being generated across the membrane entering the recirculation circuit.

5. The system of claim 4, wherein the flow of ultrafiltrate along the membrane in the recirculation circuit is at least 2, 3, 4, 5, 6, 7, 8, 9, or 10 times greater than that of flow of ultrafiltrate being generated across the membrane.

6. The system of claim 5, wherein a convectional cross flow is induced by active filtration achieved by an ultrafiltration pump, pumping ultrafiltrate out of the recirculation conduit back into the blood.

7. The system of claim 6, wherein the ultrafiltrate is pumped at a flow rate of up to 50%, 30% or 25% of the flow rate of the blood.

8. The system of claim 2, wherein the semipermeable membrane has a cut-off value of less than about 1,000,000 Da, 500,000 Da or 120,000 Da.

9. The system of claim 2, wherein the recirculation circuit comprises an active cartridge containing active cells operative to effectuate a treatment of the ultrafiltrate.

10-29. (canceled)

30. An extracorporeal detoxification system, comprising:
(a) a blood circuit configured to be coupled to a patient and operative to communicate blood from the patient, through an ultrafiltrate generator, and back to the patient;
(b) a recirculation circuit coupled to the ultrafiltrate generator and operative to draw ultrafiltrate from the ultrafiltrate generator and to treat ultrafiltrate independently of cellular components of the blood;
(c) a conduit junction operative to recombine the ultrafiltrate in the recirculation circuit and the cellular components in the blood circuit prior to reintroduction to the patient; and
(d) an albumin detoxifying component (ADC) operable to reduce albumin bound toxins and increase albumin binding capacity (ABiC).

31. The system of claim 30, wherein the ADC is operable to increase ABiC by at least 50, 55, 60, 65, 70, 75, 80, 85, 90, 95 or 100% or greater as compared to ABiC before flow through the ADC.

32. The system of claim 30, wherein the ADC is operable to reduce total concentration of bile acids to less than 50 μmol/l, 40 μmol/l, 30 μmol/l, 20 μmol/l or 10 μmol/l.

33. The system of claim 30, wherein the ADC is disposed within the blood circuit is upstream of the ultrafiltrate generator.

34. The system of claim 30, wherein the ADC is disposed within the recirculation circuit.

35. The system of claim 34, wherein the recirculation circuit comprises an active cartridge containing active cells operative to effectuate a treatment of the ultrafiltrate.

36. The system of claim 35, wherein the ADC is upstream of the active cartridge.

37. The system of claim 35, wherein the active cells are human hepatoblastoma cells.

38. The system of claim 37, wherein the active cells are C3A cells.

39-63. (canceled)

64. An extracorporeal detoxification system, comprising:
(a) a blood circuit configured to be coupled to a patient and operative to communicate blood from the patient, through an ultrafiltrate generator, and back to the patient;
(b) a recirculation circuit coupled to the ultrafiltrate generator and operative to draw ultrafiltrate from the ultrafiltrate generator and to treat ultrafiltrate independently of cellular components of the blood;
(c) a conduit junction operative to recombine the ultrafiltrate in the recirculation circuit and the cellular components in the blood circuit prior to reintroduction to the patient;
(d) a citrate infusion port; and
(e) a citrate removal component operable to remove citrate from solution.

65. The system of claim 64, wherein the citrate infusion port is in the blood circuit upstream of the ultrafiltrate generator.

66. The system of claim 65, wherein the citrate removal component is disposed in the recirculation circuit.

67. The system of claim 66, wherein the recirculation circuit comprises an active cartridge containing active cells operative to effectuate a treatment of the ultrafiltrate.

68. The system of claim 67, wherein the citrate removal component is upstream of the active cartridge.

69. The system of claim 68, wherein citrate is removed from ultrafiltrate before being processed through the active cartridge.

70. The system of claim 64, wherein the citrate removal component is a dialyzer operable to remove citrate or a citrate absorption device.

71. The system of claim 64, wherein the ultrafiltrate generator comprises a semipermeable membrane.

72. The system of claim 71, wherein the sieving coefficient for fibrinogen of the membrane is less than about 30%, 20% or 10%.

73. The system of claim 72, wherein blood from the patient is anticoagulated with citrate at a rate to maintain post membrane citrate concentrations to less than about 0.8 mmol/l, 0.5 mmol/l or 0.35 mmol/l.

74. The system of claim 73, wherein citrate is removed from ultrafiltrate before being processed through an active cartridge in the recirculation circuit.

75. The system of claim 64, further comprising a sensor for detecting ionized calcium.

76. The system of claim 75, further comprising an ionized calcium infusion port.

77. The system of claim 76, wherein the sensor and the ionized calcium infusion port are downstream of the component operable to remove citrate.

78. The system of claim 67, wherein the active cells are human hepatoblastoma cells.

79. The system of claim 78, wherein the active cells are C3A cells.

80-99. (canceled)

100. A method of performing extracorporeal detoxification comprising circulating blood of a subject through the device according to claim 1.

101. A method of treating a liver disorder or disease in a subject comprising circulating blood from the subject through the device according to claim 1 and reintroducing the blood into the subject.
